(19) United States
(12) Patent Application Publication
(10) Pub. No.: US 2013/0177520 A1
(43) Pub. Date: Jul. 11, 2013

(54) TASTE MASKED DOSAGE FORMS OF BITTER TASTING ANTI-RETROVIRAL DRUGS

(71) Applicants: Vasu Kumar Kakumanu, Guntur (IN); Shashikanth Isloor, Shimoga (IN); Vinod Kumar Arora, Gurgaon (IN)

(72) Inventors: Vasu Kumar Kakumanu, Guntur (IN); Shashikanth Isloor, Shimoga (IN); Vinod Kumar Arora, Gurgaon (IN)

(73) Assignee: RANBAXY LABORATORIES LIMITED, New Delhi, Delhi (IN)

(21) Appl. No.: 13/519,025

(22) PCT Filed: Dec. 23, 2010
(86) PCT No.: PCT/IB2010/056065

§ 371 Date: Dec. 19, 2012

(30) Foreign Application Priority Data
Dec. 31, 2009 (IN) 2757/DEL/2009

(51) Int. Cl. A61K 47/48

(52) U.S. Cl. CPC A61K 47/48184
USPC 424/78.1; 521/32

(57) ABSTRACT

The present invention relates to taste masked dosage forms of bitter tasting anti-retroviral drugs comprising a complex of the said anti-retroviral drug and an ion-exchange resin and one or more of other pharmaceutically acceptable excipients. It further relates to the processes for the preparation thereof.

### TECHNICAL FIELD OF THE INVENTION

The present invention relates to taste masked dosage forms of bitter tasting anti-retroviral drugs and to the processes for the preparation thereof.

### BACKGROUND OF THE INVENTION

Oral administration constitutes the preferred route of administration for a majority of drugs. However, several drugs, including the anti-retroviral drugs have undesirable or bitter taste which leads to lack of patient compliance in case of orally administered dosage forms. This is especially true in case of formulations intended for pediatric use. Most of the formulations of anti-retroviral drugs are solid dosage forms for adults with only a few liquid dosage forms suitable for children. Bad taste leads to poor adherence in children, which in turn causes treatment failure. Taste masking can prove to be an essential tool to improve patient compliance.

Thus, there is a need to explore ways to make it easier for the children to take anti-retroviral drugs, since these are long term chronic medications and treatment adherence is an important requisite. To address this, the present inventors have developed taste masked dosage forms of the bitter-tasting anti-retroviral drugs with an acceptable level of palatability using a hitherto unknown technique, which involves the use of ion exchange resins.

### SUMMARY OF THE INVENTION

In one general aspect, it relates to a drug-resin complex comprising a bitter tasting anti-retroviral drug and an ion-exchange resin.

In another general aspect, it relates to a drug-resin complex comprising a bitter tasting anti-retroviral drug and an ion-exchange resin, wherein the ion-exchange resin is a cation exchange resin or an anion exchange resin.

In another general aspect, it relates to a process for the preparation of a drug-resin complex comprising a bitter tasting anti-retroviral drug and an ion-exchange resin, wherein the process comprises the steps of mixing the ion-exchange resin with the drug solution/dispersion; optionally, followed by filtration/centrifugation/decantation of the drug-resin complex, dispersion, and subsequent drying by suitable method.

In another general aspect, it relates to a process for the preparation of a drug resin complex comprising a bitter anti-retroviral drug and an ion-exchange resin, wherein the process comprises the steps of passing a solution of drug through the column of ion-exchange resin; optionally, followed by filtration/centrifugation/decantation of the drug-resin complex dispersion and subsequent drying by suitable method.

In another general aspect, it relates to a dosage form comprising a drug-resin complex comprising a bitter anti-retroviral drug and an ion-exchange resin and one or more of other pharmaceutically acceptable excipients.

In another general aspect, it relates to a dosage form comprising a drug-resin complex comprising a bitter tasting anti-retroviral drug and an ion-exchange resin and one or more of other pharmaceutically acceptable excipients, wherein the dosage form is a solid dosage form or a liquid dosage form.

### DETAILED DESCRIPTION OF THE INVENTION

Anti-retroviral drugs are prescribed for the treatment of acquired immune deficiency syndrome in both adults and children. Majority of these drugs are available for administration by oral route suitable for adults. However, bitter taste in a drug is an inherent disadvantage with certain types of oral preparations, particularly when intended for use in pediatric population. Therefore, there is a need to develop taste masked dosage forms of these bitter tasting anti-retroviral drugs that can result in better patient compliance and increase the chance of treatment adherence and success, particularly in case of children.

Various techniques are known for masking the bitter taste of drugs, including taste-masking with excipients such as flavors, sweeteners, gelatin, gelatinized starch, surfactants, lecithins and lecithin-like substances, salts, polymeric membranes etc.; taste masking polymer coating; taste masking by conventional granulation; taste masking by spray congealing with lipids; taste masking by ion-exchange resins; taste masking by formation of inclusion complexes with cyclodextrins; taste masking by the freeze-drying process; taste masking by multiple emulsions etc. Surprisingly, the present inventors have found that the bitter taste of the resulting formulation can be substantially eliminated by complexing the bitter tasting anti-retroviral drugs with suitable ion-exchange resins. With the correct selection of the ion-exchange resin, the drug-resin complex does not break at pH of saliva and the drug is not released in the mouth. When the drug-resin complex comes in contact with the gastro-intestinal fluids, for example, the acid of the stomach, the drug is released from the complex and absorbed in the usual way.

“Ion-exchange resin”, as recited herein implies water-insoluble polymers that contain acidic or basic functional groups and have the ability to exchange counter-ions with aqueous solutions surrounding them. It includes cationic or anionic resins. The cation exchange resins include, but are not limited to, sulphonated copolymers of styrene and divinylbenzene (e.g. Sodium polystyrene sulphonate), polystyrene matrix cation exchange resins, copolymers of methacrylic acid and divinylbenzene (e.g. polacrilex) and cross-linked polymers of methacrylic acid and divinylbenzene (e.g. Polacrilin potassium) such as those available commercially as Dowex® resins, Amberlite® IRP resins, Tulsion® resins, Indion resins and their equivalents in acid form or in the form of salt with alkali metals. Anion exchange resin include but are not limited to, quarternized amine resins resulting from the reaction of triethylamine with chloromethylated copolymer of styrene and divinylbenzene, primary or secondary amine resins with chloromethylated copolymer of styrene and divinylbenzene and cholestyramine resin USP (commercially available as Duolite®). Ion-exchange resins may comprise from about 0.01% to about 95% by weight of the pharmaceutical compositions described herein.

Generally, two techniques may be used to prepare the drug-resin complexes described in the present application. In the first method, the drug-resin complex may be prepared by mixing the specific quantity of ion-exchange resin with the drug solution until the equilibrium is established. In the second method, the drug-resin complex is formed by passing a solution of drug through the column of ion-exchange resin until the effluent concentration is the same as the eluent concentration. The resultant drug-resin complex dispersion can be suspended directly into suitable vehicles to be formulated into liquid dosage forms or can be concentrated by decantation procedure prior to suspension. Alternatively, the solids are separated by filtration/centrifugation/decantation or by combination of these techniques and then dried to be formulated into solid dosage forms. Process of drying may be selected from evaporation, vacuum evaporation, tray drying, fluid bed dryer, oven drying, air drying at room or elevated temperatures, microwave drying, spray drying, drum and belt film drying; or by centrifuging or by any other suitable method.

The complexes of the anti-retroviral drug and the ion-exchange resin may be formulated as either solid dosage forms or liquid dosage forms. These may include without limitation, tablets; chewable tablets; mouth dissolving tablets; dispersible tablets; sprinkles; granules/powders/pellets for filling into sachets/capsules/bottles; or solutions; suspensions; syrups and the like.

The dosage forms may further comprise one or more of other pharmaceutically acceptable excipients depending on the dosage form to be formulated. Pharmaceutically acceptable excipients may include, without limitation, diluents; binders; lubricants/glidants; disintegrants; buffer systems; surfactants; preservatives; thickening/suspending agents; sweetening agents; flavoring agents; coloring agents; solvents/co-solvents and the like.

Suitable diluents that may be used include, but are not limited to, microcrystalline cellulose, silicified microcrystalline cellulose, microfine cellulose, lactose, starch, pregelatinized starch, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, as well as other conventional diluents well known to the persons skilled in the art.

Suitable binders that may be used include, but are not limited to, acacia, guar gum, alginic acid, carbomer, dextrin, maltodextrin, methylcellulose, ethyl cellulose, hydroxyethylcellulose, hydroxypropylcellulose, hydroxypropylmethylcellulose, carboxymethylcellulose sodium, magnesium aluminum silicate, polymethacrylates, crospovidones, povidones, copovidones, gelatin, starch, as well as other conventional binders well known to the persons skilled in the art.

Suitable lubricants/glidants that that may be used include, but are not limited to, magnesium stearate, zinc stearate, calcium stearate, stearic acid, colloidal silicon dioxide, glyceryl palmitostearate, vegetable oils, polyethylene glycols, polyvinyl alcohols, talc, sodium benzoate, sodium stearyl fumarate, magnesium oxide, poloxamer, sodium lauryl sulphate, polyoxyethylene monostearate, cocoa butter, hydrogenated vegetable oils, mineral oil, polysaccharides as well as other conventional lubricants/glidants well known to the persons skilled in the art.

Suitable disintegrants that may be used include, but are not limited to, mannitol, alginic acid, carboxymethylcellulose, hydroxypropylcellulose, microcrystalline cellulose, croscarmellose sodium, crospovidone, magnesium aluminum silicate, methylcellulose, povidone, sodium alginate, sodium starch glycolate, starch, as well as other conventional disintegrants well known to the persons skilled in the art.

Suitable buffer systems include, but are not limited to, sodium hydroxide, acetic, boric, carbonic, phosphoric, succinic, malaic, tartaric, citric, benzoic, lactic, glyceric, gluconic, glutaric and glutamic acids and their sodium, potassium and ammonium salts, as well as other conventional buffer systems well known to the persons skilled in the art.

Suitable surfactants include, but are not limited to, polyoxyethylene sorbitan fatty acid esters, polyoxyethylene monoalkyl ethers, sucrose monoesters and lanolin esters and ethers, alkyl sulfate salts, sodium, potassium, and ammonium salts of fatty acids, as well as other conventional surfactants well known to the persons skilled in the art.

Suitable preservatives include, but are not limited to, phenol, esters of hydroxybenzoic acid, sorbic acid, o-phenylphenol benzoic acid and the salts thereof, chlorobutanol, benzyl alcohol, thimerosal, phenylmercuric acetate and nitrate, nitromersol, benzalkonium chloride, cetylpyridinium chloride, methyl paraben, and propyl paraben as well as other conventional preservatives well known to the persons skilled in the art.

Suitable thickening/suspending agents include, but are not limited to, methylcellulose, sodium carboxymethylcellulose, hydroxypropylmethylcellulose, hydroxypropylcellulose, sodium alginate, carbomer, povidone, acacia, guar gum, xanthan gum, gum tragacanth, locust bean gum, microcrystalline cellulose, colloidal silicas, as well as other conventional thickening/suspending agents well known to the persons skilled in the art.

Additional taste-masking agents that may be used include flavors and sweeteners. Flavors may be chosen from natural and synthetic flavor liquids and include, but are not limited to, volatile oils, synthetic flavor oils, flavoring aromatics, oils, liquids, oleoresins or extracts derived from plants, leaves, flowers, fruits, stems and combinations thereof. The sweeteners may be chosen from the following non-limiting list: sucrose, dextrose, invert sugar, fructose, and mixtures thereof, saccharin, aspartame, acesulfame, sucralose, sugar alcohols such as sorbitol, mannitol, xylitol, and the like.

Suitable coloring agents include, but are not limited to, titanium dioxide pigments, lake colors, iron oxide pigments, and the like.

Suitable solvents and/or co-solvents that may be used for several purposes include, but are not limited to, water, ethanol, organic polar and non-polar solvents, glycerin, propylene glycol, polyethylene glycol and their suitable mixtures.

Dosage forms comprising the drug-resin complexes as prepared herein may be made by conventional processes in the art using the commonly available equipment.

Granules for direct ingestion or for reconstitution before administration may be prepared by granulating the drug-resin complex as prepared herein with one or more of other pharmaceutically acceptable excipients.

Alternatively, the drug-resin complex as prepared herein may be mixed with one or more of other pharmaceutically acceptable excipients and then filled into bottles for use as reconstitutable powder or filled in capsules.

Tablets comprising the drug-resin complex may be prepared by the conventional processes of the wet or dry granulation or direct compression.

Liquid dosage forms such as solutions, suspensions, or syrups may be obtained by dispersing the drug-resin complex in a suitable vehicle such as water or any other suitable solvent and optionally adding other pharmaceutically acceptable excipients; and filling the resultant solution, suspension, or syrup into bottles.

Anti-retroviral drugs for preparing the drug-resin complexes may include drugs having an inherent bitter taste and include without limitation, tenofovir, abacavir, didanosine, lamivudine, emtricitabine, stavudine, zidovudine, ritonavir, atazanavir, darunavir, indinavir, lopinavir, saquinavir, tipranavir, nelfinavir, amprenavir, fosamprenavir and the like. The dosage forms may also comprise more than one anti-retroviral drug. In that case, the drugs may be individually complexed with the resin or they may be mixed together and the mixture of anti-retroviral drugs is then complexed with a resin. The resultant drug-resin complexes are then formulated into a suitable dosage form.

Preferred pharmaceutical compositions of the present invention may take form of several different embodiments:

In one embodiment, it relates to a drug-resin complex comprising a bitter tasting anti-retroviral drug selected from tenofovir disoproxil fumarate, lamivudine, abacavir, sulphate, emtricitabine, stavudine, lopinavir, ritonavir and atazanavir sulphate and polacrilex as the ion-exchange resin.

In another embodiment, it relates to a drug-resin complex comprising a bitter tasting anti-retroviral drug selected from emtricitabine, stavudine, lopinavir, ritonavir, and atazanavir sulphate and polacrilin potassium as the ion-exchange resin.

In another embodiment, it relates to a drug-resin complex comprising a bitter tasting anti-retroviral drug selected from lamivudine, abacavir sulphate, emtricitabine, stavudine, lopinavir, and ritonavir and sodium polystyrene sulphonate as the ion-exchange resin.

In the above embodiments, the process for the preparation of the drug-resin complex may comprise the steps of:
1. Adding the ion-exchange resin in a sufficient quantity of distilled water to make about 10% slurry and stiffing for about 20 minutes to obtain a uniform, lump-free dispersion.
2. Mixing the drug with the above dispersion slowly under stiffing to obtain the drug:resin ratio of 1:3.
3. Stirring the entire mixture for about 4 hours which is then set aside for settling.
4. The above dispersion is either used as such for formulating into liquid dosage forms or subjected to one of the below processes:
   a) The obtained dispersion is concentrated by decantation for formulating into liquid dosage forms; or
   b) The obtained dispersion is filtered and dried in tray-dryer for formulating into solid dosage forms.

In another embodiment, it relates to a dispersible tablet comprising a drug-resin complex prepared as per the above embodiment and one or more of pharmaceutically acceptable excipients, wherein the drug is tenofovir disoproxil fumarate and the resin is polacrilex.

In another embodiment, it relates to a powder for reconstitution comprising a drug-resin complex prepared as per the above embodiment and one or more of other pharmaceutically acceptable excipients, wherein the drug is tenofovir disoproxil fumarate and the resin is polacrilex.

In another embodiment, it relates to a suspension comprising a drug-resin complex prepared as per the above embodiment and one or more of other pharmaceutically acceptable excipients, wherein the drug is ritonavir and the resin is polacrilex.

The following examples are given for the purpose of illustrating the present invention and are not intended to limit the scope in any way:

### EXAMPLES

### Example 1

### Taste Masked Resin Complexes of Some of the Bitter Tasting Anti-Retroviral Drugs

| Drug Name | Resins used |
|---|---|
| Tenofovir disoproxil fumarate | Polacrilex |
| Lamivudine | Polacrilex/Sodium polystyrene sulphonate |
| Abacavir sulphate | Polacrilex/Sodium polystyrene sulphonate |
| Emtricitabine | Polacrilex/Polacrilin potassium/Sodium |
| | polystyrene sulphonate |
| Stavudine | Polacrilex/Polacrilin potassium/Sodium |
| | polystyrene sulphonate |
| Lopinavir | Polacrilex/Polacrilin potassium/Sodium |
| | polystyrene sulphonate |
| Ritonavir | Polacrilex/Polacrilin potassium/Sodium |
| | polystyrene sulphonate |
| Atazanavir sulphate | Polacrilex/Polacrilin potassium |

### Procedure for the Preparation of Drug-Resin Complexes:

1. 9 grams of ion-exchange resin was added to sufficient quantity of distilled water to make about 10% slurry and stirred for 20 minutes to obtain a uniform, lump-free dispersion.
2. 3 grams of drug was added to the above dispersion, slowly under stirring.
3. The entire mixture was stirred for about 4 hours and then set aside for settling.
4. The above dispersion was either used as such for formulating into liquid dosage form or subjected to one of the below processes:
   a) The obtained dispersion was concentrated by decantation for formulating into liquid dosage form; or
   b) The obtained dispersion was filtered and dried in tray-dryer for formulating into a solid dosage form.

### Example 2

### Taste Masked Dispersible Tablets Comprising Tenofovir-Resin Complex

| Ingredient | Quantity (in mg) per tablet |
|---|---|
| Tenofovir-Polacrilex Complex | 280.00 |
| Polyplasdone XL | 20.00 |
| Aspartame | 15.00 |
| Acesulfame potassium | 7.00 |
| Microcrystalline cellulose | 72.00 |
| Magnesium stearate | 3.00 |
| Mint flavour | 3.00 |
| Total Weight | 400.00 |

### Procedure:

1. Tenofovir-Polacrilex complex prepared as per the procedure in Example 1 and all other excipients were sifted through a suitable sieve.
2. All the ingredients were mixed for about 5 minutes to get a uniform mixture.
3. The resultant blend was compressed into suitable sized tablets using appropriate tooling.

### Example 3

### Taste Masked Reconstitutable Powder Comprising Tenofovir-Resin Complex

| | Quantity (in mg) per 5 ml |
|---|---|
| Ingredient | after reconstitution |
| Tenofovir-Polacrilex Complex | 280.00 |
| Xanthan Gum | 25.00 |
| Aspartame | 15.00 |
| Acesulfame potassium | 7.00 |
| Xylitol | 25.00 |
| Silicon dioxide | 5.00 |
| Mint flavour | 3.00 |
| Total Weight | 360.00 |

### Procedure:

1. Tenofovir-Polacrilex complex prepared as per the procedure in Example 1 and all other excipients were sifted through a suitable sieve.
2. All the ingredients were mixed for about 5 minutes to get a uniform mixture.
3. The resultant blend was filled into bottles by suitable means.

### Example 4

### Taste Masked Suspension Comprising Ritonavir-Resin Complex

| Ingredient | Quantity (in mg) per 5 ml |
|---|---|
| Ritonavir-Polacrilex Complex | 200.00 |
| Xanthan Gum | 25.00 |
| Sucralose | 25.00 |
| Xylitol | 25.00 |
| Silicon dioxide | 5.00 |
| Mint flavour | 3.00 |
| Total weight | 283.00 |

### Procedure:

1. All the excipients were sifted through a suitable sieve.
2. The sifted excipients were added to Ritonavir-Polacrilex suspension prepared as per the procedure in Example 1.
3. The resultant suspension was filled into bottles by suitable means.

We claim:

1. A drug-resin complex comprising a bitter tasting anti-retroviral drug and an ion-exchange resin.

2. The drug-resin complex according to claim 1, wherein the bitter tasting antiretroviral drug comprises one or more of tenofovir disoproxil fumarate, abacavir sulphate, didanosine, lamivudine, emtricitabine, stavudine, zidovudine; ritonavir, atazanavir, darunavir, indinavir, lopinavir, saquinavir, tipranavir, nelfinavir, amprenavir, or fosamprenavir.

3. The drug-resin complex according to claim 1, wherein the ion-exchange resin is a cation exchange resin or an anion exchange resin.

4. The drug-resin complex according to claim 3, wherein the ion-exchange resin is polacrilex.

5. The drug-resin complex according to claim 3, wherein the ion-exchange resin is polacrilin potassium.

6. The drug-resin complex according to claim 3, wherein the ion-exchange resin is sodium polystyrene sulphonate.

7. A process for the preparation of a drug-resin complex according to claim 1, wherein the process comprises the steps of mixing the ion-exchange resin with the drug solution/dispersion; optionally, followed by
filtration/centrifugation/decantation of the drug-resin complex, dispersion, and subsequent drying.

8. The process for the preparation of a drug resin complex according to claim 1, wherein the process comprises the steps of passing a solution of drug through the column of ion-exchange resin; optionally, followed by
filtration/centrifugation/decantation of the drug-resin complex, dispersion, and subsequent drying.

9. A dosage form comprising a drug-resin complex of claim 1 and one or more of other pharmaceutically acceptable excipients.

10. The dosage form according to claim 9, wherein the dosage form is a solid dosage form.

11. The dosage form according to claim 9, wherein the dosage form is a liquid dosage form.

12. The dosage form according to claim 9, wherein the other pharmaceutically acceptable excipients comprise one or more of diluents, binders, lubricants, glidants, disintegrants, buffer systems, surfactants, preservatives, thickening agents, suspending agents, sweetening agents; flavoring agents; coloring agents; solvents and co-solvents.
